(19) United States
(12) Patent Application Publication
(10) Pub. No.: US 2010/0152616 A1
(43) Pub. Date: Jun. 17, 2010

(54) DEVICES AND METHODS FOR SAFELY ACCESSING BONE MARROW AND OTHER TISSUES

(76) Inventors: Niyazi Beyhan, Santa Clara, CA (US); Surag Mantri, Sunnyvale, CA (US); Hoang Nguyen, San Jose, CA (US)

Correspondence Address: D. BOMMI BOMMANNAN, 2251 Grant Road, Suite B, LOS ALTOS, CA, 94024, US

(21) Appl. No.: 12/699,847

(22) Filed: Feb. 3, 2010

Related U.S. Application Data
(63) Continuation-in-part of application No. 12/274,329, filed on Nov. 19, 2008.
(60) Provisional application No. 61/149,786, filed on Feb. 4, 2009.
(60) Provisional application No. 61/003,772, filed on Nov. 20, 2007.
(60) Provisional application No. 61/078,736, filed on Jul. 7, 2008.

(51) Int. Cl. A61B 10/00

(52) U.S. Cl. 600/568; 600/567

(57) ABSTRACT

A device for safely accessing bone marrow and other tissues is disclosed. Such device comprises a needle assembly, a sensor mechanism, and an actuator configured to engage with the needle assembly via the sensor mechanism to advance a tissue penetrable needle through a first tissue region and into a second tissue region. In one aspect, the first tissue region is a bone cortex and the second tissue region is a bone marrow cavity. The sensor mechanism is configured to disengage the actuator from the needle once the needle has crossed the first tissue region (e.g., bone cortex) into the second tissue region (e.g., bone marrow cavity), thereby preventing the needle from advancing further into the second tissue region. In one aspect, the sensor mechanism is mechanical and comprises a pin assembly comprising a spring-loaded pin that extends through the needle. The pin assembly is configured such that advancement of the distal tip of the spring-loaded pin past the distal tip of the needle causes the actuator to disengage from the needle. The actuator is a rotatable actuator. A catheter-based embodiment of the device can be inserted in body passageways to access tissue internally within the body.

### CROSS-REFERENCES TO RELATED APPLICATIONS

This application is a continuation in part of U.S. patent application Ser. No. 12/274,329, filed on Nov. 19, 2008, which claims priority to U.S. Provisional Patent Application No. 61/003,772 filed on Nov. 20, 2007, and to U.S. Provisional Patent Application No. 61/078,736 filed on Jul. 7, 2008. This application also claims priority to U.S. Provisional Patent Application No. 61/149,786 filed on Feb. 4, 2009.

### FIELD OF THE INVENTION

The present invention pertains generally to the field of medical instruments, and more particularly to those instruments for safely accessing bone marrow and other tissues.

### DESCRIPTION OF THE RELATED ART

### Some Uses of Biopsy, Aspiration, and Intraosseous Infusion

Access to the bone marrow or the bone marrow cavity is often necessary for a wide variety of diagnostic and therapeutic procedures that involve biopsy, aspiration, and intraosseous infusion. For example, a biopsy may require penetrating the bone marrow cavity and withdrawing bone marrow or other bone marrow cavity fluids for a diagnostic study or for obtaining sufficient quantity of bone marrow to be used in transplant procedures. Such biopsy specimens can be used to diagnose a wide variety of diseases, including many forms of cancer for which fluid, tissue and bone marrow biopsies are widely used.

As a particular example, new research indicates that micrometastasis of breast cancer in bone marrow aspirant might be an important predictor for recurrence and long term survival, requiring those patients to be treated differently than those who do not have micrometastasis of breast cancer in their bone marrow aspirant. There are many other examples of the use of bone marrow, such as using bone marrow aspirants in stem cell research and other advanced clinical and laboratory research today. It will thus be appreciated that the quick retrieval of such specimens with minimal discomfort and trauma to the patient is important to the success of such procedures. A sternal puncture to obtain bone marrow aspirant during lumpectomy and sentinel node biopsy procedures may offer convenient access with less pain inflicted on the patient.

Access to bone marrow and bone marrow cavity is also important in procedures that involve intraosseous infusion. Intraosseous infusion provides an alternative route for the administration of fluids and medications when it is difficult to instill peripheral or central lines during resuscitation of the critically ill or injured patients, or during neo-natal care. The sternum is a likely site of choice for intraosseous infusion for many reasons: 1) the sternal body is large and relatively flat, and can be readily located by unskilled practitioners; 2) the sternum retains a high proportion of red marrow; 3) the sternum has a thinner, more uniform cortical bone covering overlying a relatively uniform marrow space; and 4) the sternum (particularly the manubrium) is less likely to be fractured than the extremities.

Additionally, any procedure that involves sternotomy (such as open heart surgery, etc.) may potentially infect the sternum of the patient. Therefore, it would be desirable to safely and easily obtain cytology samples (i.e., bone marrow aspirant) post surgery in order to examine whether the sternum is healing without infections.

Today, biopsy specimens can be obtained by surgical excision or needle biopsy procedures. In general, a needle biopsy procedure involves inserting a cannula-and-stylet assembly through an incision until the tip of the assembly is at or near the site from which a biopsy specimen is desired. After positioning of the assembly, the stylet is withdrawn and the cannula is inserted further to collect a specimen in its distal end. Suction is then applied to the proximal end of the cannula, usually by means of a conventional syringe, to obtain or assist in collecting the specimen for subsequent histological and/or cytological examination in a laboratory. The most preferred site for bone marrow aspiration today is the upper iliac crest, despite the fact that the sternum is the most convenient location. This is because many bone marrow aspiration and biopsy needles designed for sternal access, such as that disclosed in U.S. Pat. No. 6,761,726, are still not sufficiently safe and do not sufficiently guard against over penetration, despite the fact that they provide a pre-adjustable spacer to prevent accidental penetration of the posterior wall of the cortex. These devices thus rely on the physician's judgment of the thickness of the sternum, which can vary widely from obese to pediatric cases. Due to the risk of perforating the aorta or creating cardiac tamponade with devices that are not completely safe, most physicians prefer to obtain bone marrow aspirants through the upper iliac crest.

Pyng Medical Corporation from Canada provides an intraosseous drug infusion device which registers the depth of the needle penetration from the anterior surface of the cortex and thus brings some safety to the patients who are injured or critically ill. However, this system does not take into account the variations in the sternum, cortex or marrow thickness and therefore provides limited safety. Additionally, the device is not suitable for conditions that are not immediately life threatening, such as a routine bone marrow aspiration, since the risk of penetrating the organs underlying the sternum is justified only in the case of life threatening situations. Other devices from companies, such as Vidacare, prefer infusion into the tibia using special drill-like drivers.

A variety of biopsy needle units have been available in the market. In general, such units include an outer cannula or hollow needle, with a removable inner needle and/or solid styles extending there-through. Some releasable locking means is generally provided for securing the inner needle and/or the stylus in a longitudinal position within the outer needle.

U.S. Pat. No. 4,314,565 discloses a biopsy needle unit, wherein the needle is removably secured by a threaded collar in the chuck of a generally T-shaped stainless steel handle. This unit utilizes a replaceable needle, which can be sterilized and sharpened for reuse, or disposed of after use, but the handle still requires sterilization after each use.

Several devices exist in which mechanical detection of penetration is used, for example as disclosed in U.S. Pat. Nos. 5,226,426; 5,318,585; and 5,817,052. However, these devices rely on complicated mechanical systems for their functioning. Additionally, these devices are not disposable, adding the complexity of sterilization and associated risks.

Completely disposable biopsy needle units have been available; however, these have not been altogether satisfactory. Such units tend to incorporate stainless steel needles with plastic handles, which in turn is a critical stress junction. Breakage or slippage at this junction during insertion of the needle can cause injury to the patient. Similarly, breakage or slippage at the junction between the solid stylet and its end cap can cause injury to the doctor performing the procedure. It should be appreciated that considerable pushing and twisting forces are applied to such devices during use, particularly while obtaining bone marrow specimens, and devices with low stress junctions or that are easily breakable are unsafe.

Some of the disposable biopsy needle units of the prior art have been of three-piece construction with a separate cover cap for securing the stylet in place; the cap can be difficult to remove, particularly with gloved hands. For example, disposable bone marrow biopsy/aspiration needles of this type are available from the Monoject division of Sherwood Medical of St. Louis, Mo. U.S. Pat. No. 4,258,722 is also representative of the existing technology in this regard.

Other biopsy needle units of the prior art have been of two-piece construction, but with other drawbacks. For example, U.S. Pat. No. 4,469,109 discloses such a two-piece unit; however, the stylet is secured to the needle by means of a button-and-spring detent locking-groove that requires a twisting motion to engage or disengage, which can be inconvenient, if not awkward, with gloved hands. A converging bore is provided for receiving the end of a syringe for aspiration, but no means are provided for quickly and conveniently securing the syringe and needle together. As a result, the physician usually requires an assistant to complete the procedure. A need has thus arisen for an improved disposable biopsy needle unit of inexpensive but safe, secure, and reliable construction, which is also comfortable and easy to manipulate by one person.

Some bone marrow biopsy needles are used to obtain bone marrow samples for diagnostic purposes as well as for harvesting marrow for transplant purposes. Such biopsy needles generally include a cannula member having a stainless steel cannula with a hub or handle connected to the proximal end of the cannula, and a stylet member having a stainless steel stylet and a handle or cap connected to the proximal end of the stylet. The cannula is provided with a sharp distal end and receives the stylet. The stylet is provided with a sharp pointed distal end that extends beyond the distal end of the cannula when the cannula and stylet members are assembled together for penetrating body tissue and bone. This allows the stylet to enter a bone marrow cavity, such as the iliac crest of the patient. Both handles are hand grasped and considerable pressure is applied to cause the distal ends of the cannula and stylet to penetrate the tissue and bone.

Aspiration of a bone marrow sample is accomplished by removing the stylet member from the cannula member while the distal end of the cannula is in the marrow cavity. A syringe is then connected to the proximal end of the cannula, and marrow fluid is aspirated into the syringe. The aspirated sample is then processed for clinical testing. Where harvesting of marrow, such as for a transplant, is desired, a relatively large number of biopsy needle insertions and marrow aspirations are generally required to collect a suitable amount of marrow. Where a biopsy core sample is to be obtained, the distal ends of the cannula and stylet are inserted into the marrow cavity and, after the stylet is removed, the cannula handle is rotated back and forth while applying axial pressure to the cannula to move the cannula through the marrow and collect a sample core within the cannula. The cannula is then carefully removed from the patient and the core sample pushed through the cannula and out the proximal end, such as by employing a probe. Aspirated samples and core samples may be taken from the same patient for a more complete diagnosis.

Good manual control of the biopsy device during insertion into the bone is necessary to avoid inadvertent injury to the patient. The size and shape of the upper portion of the biopsy device that is grasped by the hand of the practitioner, both with and without the stylet member in place, are important factors in providing good control of the device during use. Some biopsy devices have had handle portions which tend to concentrate the reaction forces during insertion to relatively small areas of the hand. This tends to produce discomfort or even injury to the practitioner and less control of the biopsy device because of the relatively high pressures applied to the device. This is especially the case where a considerable amount of bone marrow must be collected, such as in the case of a transplant, and where a relatively large number of biopsies are required.

Some biopsy devices employ a ball-type handle which engages a relatively small middle portion of the palm of the hand or a handle having protuberances which tend to concentrate the reaction force to relatively small areas of the palm. Some such devices, in general, have been uncomfortable to the practitioner especially where repeated samples need to be obtained. In some cases, when the stylet member is removed from the cannula member, the remaining handle of the cannula member has a shape or protuberances that produce discomfort to the practitioner.

Some such biopsy devices have handle locking constructions which produce undesirable protuberances that are in contact with the hand in use, or constructions which cannot readily use a Luer lock connector for connecting the cannula to a syringe tip Luer lock for aspiration of bone marrow fluid. In penetrating the hard, outer layer of marrow-containing bones, a sharpened surgical instrument such as a stylet, often fittingly mated within a cannula, is generally used. The instrument must be designed to allow the physician to exercise both the necessary pressure to penetrate the hard outer layer of the bone, as well as extreme care to avoid unnecessary damage to bone and surrounding tissue. During the penetration procedure, the instrument should allow proper handling to avoid slipping on the outer surface of the bone and to allow proper positioning and orientation of the instrument, particularly when the bone marrow cavity is to be reached. Previous biopsy needles have presented disadvantages when used in this procedure. Often the handle does not allow maintaining a secure grip on the instrument, while also controlling the orientation of the stylet and cannula during the twisting and penetrating forces exercised in the bone penetration procedure. Examples of previously available biopsy needles are those described in U.S. Pat. Nos. 4,141,365; 4,256,119; 4,262,676; 4,326,519; 4,487,209; 4,513,754 and 4,747,414. The commonly assigned U.S. Pat. Nos. 4,403,617 and 4,630,616 disclose a bone biopsy needle having a cannula and a stylet which is slidably received within the lumen of the cannula. The stylet has a handle rigidly attached to its proximal end, the handle having a broad, palm-contacting surface extending at right angles on either side of the axis of the stylet. The cannula has a handle-receiving recess designed to prevent rotation of the handle about the stylet axis when the handle and the handle-receiving means are mated together.

U.S. patent application Ser. No. 11/332,493 discloses detection of needle penetration using an accelerometer sensor. However, this device suffers from the drawback that the system is both costly and not disposable.

Percutaneous vertebroplasty is another area where a more controlled access to the interior of a vertebral body is desirable. The general procedure for performing percutaneous vertebroplasty involves the use of a standard 11 gauge Jamshidi needle. The needle includes an 11 gauge cannula with an internal stylet. The cannula and stylet are used in conjunction to pierce the cutaneous layers of a patient above the hard tissue to be supplemented, then to penetrate the hard cortical bone of the vertebra, and finally to traverse into the softer cancellous bone underlying the cortical bone.

During this procedure, a large force must be applied by the user, axially through the Jamshidi needle, in order to drive the stylet through the cortical bone. Once penetration of the cortical bone is achieved, additional downward axial force, albeit at a reduced magnitude compared to that required to penetrate the cortical bone, is required to position in the stylet/tip of the cannula into the required position within the cancellous bone. If the force magnitude is not reduced appropriately, or if very soft bone is encountered, as is often the case with osteoporotic patients, the stylet and cannula can be accidentally and suddenly drive through the cortical bone on the opposite side of the vertebra. This is a very dangerous and potentially lethal situation in the case of vertebroplasty, since the aorta is located in close proximity to the anterior surface of at least the thoracic and lumbar vertebrae, and could easily be punctured by such an occurrence. Additionally, with regard to all vertebrae, the spinal cord is located medially of the pedicle, and could also be damaged by a piercing stylet. Accordingly, there exists a need for a more controlled approach to the interior of a vertebral body for the performance of vertebroplasty, and particularly, for percutaneous vertebroplasty.

Accessing spaces in the sinus is another area where a more controlled approach to the interior is desirable. For example, when the sinuses are infected, evacuation of purulent secretion is often desirable and sometimes necessary. During the evacuation procedure, typically a needle (approximately 1.4 mm) is placed at the penetration site under the inferior concher in the nose whereupon the needle is advanced through the bone by steady forward pressure. A sensation of decreased resistance usually indicates penetration of the medial sinus wall. The needle is then further advanced about 2 mm into the cavity whereupon aspiration of the secretion for bacterial culture is usually performed whereupon the cavity is flushed with saline solution through the inserted needle. The pain and discomfort associated with this procedure is often substantial. As discussed in the previous methods it is often difficult to stop the motion upon penetration of the bone due to the high applied force that is required. Penetration of the second sinus wall may cause serious bleeding and complications in the orbit. In some patients the medial sinus wall is so hard that penetration is impossible with the currently available equipment. Accordingly, there exists a need for a more controlled approach to accessing the interior of a sinus.

For these reasons, it would be desirable to provide alternative and improved methods and apparatus for accessing bone marrow and other tissues. In particular, it would be desirable to provide methods, systems and devices that enable rapid sampling while being safe and easy to use by the practitioner. Such a device should also be compatible with standard syringe fittings while being inexpensive and disposable. At least some of these objectives will be met by the inventions described herein below.

### SUMMARY OF THE INVENTION

Various embodiments of a device for safely accessing bone marrow and other tissues are disclosed. The device comprises a needle assembly, a sensor mechanism, and an actuator. The needle assembly comprises a tissue penetrable needle having a distal tip. The needle comprises a bore in order to allow aspiration or infusion through the needle. The actuator is configured to engage with the needle via a pin assembly to advance the needle through a first tissue region and into a second tissue region. In one aspect, the device is configured such that the needle advances through a bone cortex (first tissue region) and into a bone marrow cavity (second tissue region). The actuator is a rotatable actuator such as a knob or a T-shaped handle.

The sensor mechanism senses a differential in a tissue region characteristic between the first and second tissue regions, and is configured to disengage the actuator from the needle once the needle has crossed the first tissue region (e.g., bone cortex) into the second tissue region (e.g., bone marrow cavity), thereby preventing the needle from advancing further into the second tissue region.

In one aspect, the sensor mechanism is a mechanical sensor mechanism which comprises a pin assembly with a spring-loaded pin that extends through the needle bore. The pin assembly is configured such that advancement of the distal tip of the spring-loaded pin past the distal tip of the needle causes the actuator to disengage from the needle. The needle assembly and the pin assembly can thereafter be withdrawn, leaving the second tissue region (e.g., bone marrow) accessible through a syringe or other medical instrument.

In another aspect, the device is configured to provide safe access to tissue or cavities in internal body parts that may be more readily approached through body passageways such as the digestive or respiratory systems of the body. In such embodiments, a flexible catheter is used for such internal access, and the needle is connected to the needle assembly through a flexible coil, while the pin is attached to the pin assembly via a flexible shaft. The needle is sheathed in a flexible catheter. The device is used to access tissue by operating the actuator at the proximal end by to advance the needle through tissue.

Other aspects of the invention include methods corresponding to the use of the devices and systems described above.

### BRIEF DESCRIPTION OF THE DRAWINGS

The invention has other advantages and features which will be more readily apparent from the following detailed description and the appended claims, when taken in conjunction with the accompanying drawings, in which:

FIG. 1 shows exemplary components of one embodiment of a device for safely accessing bone marrow and other tissue.

FIG. 2A provides a detailed view of the actuator. The knob teeth engage with the pin assembly teeth.

FIG. 2B provides a detailed view of the pin assembly, actuator and spring.

FIG. 2C provides a detailed view of the needle assembly.

FIG. 2D shows a cross-sectional view of the needle base with anchoring mechanism used to position the device upon tissue.

FIG. 3A shows a cross-sectional view of the first step of a bone aspiration procedure with starting positions of the needle assembly and actuator.

FIG. 3B shows a cross-sectional view of the second step of the bone aspiration procedure showing needle advancement into tissue.

FIG. 3C shows a cross-sectional view of the third step of the procedure showing needle advancement through the anterior bone cortex.

FIG. 3D shows a cross-sectional view of the fourth step of the procedure in which the marrow is penetrated, with the pin extending past the distal tip of the needle, thereby disengaging the actuator from the needle and preventing further advancement of the needle into the bone marrow cavity.

FIG. 4A shows post-penetration removal of the actuator to facilitate tissue access.

FIG. 4B illustrates access through the device using a syringe.

FIG. 5 shows the base after removal of the needle assembly at the end of the procedure.

FIG. 6 shows an embodiment of the device employing a modified base configuration.

FIG. 7A illustrates another embodiment of the device intended for iliac access.

FIG. 7B illustrates the use of the iliac access embodiment.

FIG. 7C further illustrates the use of the iliac access embodiment.

FIG. 7D further illustrates the use of the iliac access embodiment as it is pushed towards the iliac crest.

FIG. 7E further illustrates the use of the iliac access embodiment.

FIG. 7F shows a detailed cross sectional view of the iliac access embodiment as it references the iliac crest.

FIG. 7G shows a view of the top of the base when the iliac access embodiment has referenced the iliac crest.

FIG. 7H shows a detailed view of the top of the base when the iliac access embodiment has referenced the iliac crest.

FIG. 8A shows the catheter-based tissue access device in assembled condition.

FIG. 8B depicts the catheter-based tissue access device in disassembled condition, showing the individual parts.

FIG. 8C shows the needle and pin in cross sectional detail.

FIG. 8D shows an embodiment comprising a spring designed to limit force at the distal tip of the catheter when placed in contact with tissue.

FIG. 8E illustrates an embodiment in which a balloon is used to allow better referencing of the tissue boundary by the tip of the catheter.

FIG. 8F shows an embodiment in which a cup is used at the tip of the catheter to shape the inflation of the balloon.

FIG. 8G shows an embodiment that uses vacuum suction at the tip of the catheter.

FIG. 8H shows an embodiment that uses a balloon as well as vacuum to support and anchor the tissue to the catheter.

FIGS. 9A-9C shows an embodiment that uses a balloon at the tip of the catheter to stabilize the device within a body cavity for tissue access.

FIG. 10A shows an embodiment of the catheter that allows the invention to be used in the working channel of a viewing scope.

FIG. 10B shows another view where the tissue access catheter is shown locked in a desired position along the working channel of the viewing scope.

### DETAILED DESCRIPTION

Although the detailed description contains many specifics, these should not be construed as limiting the scope of the invention but merely as illustrating different examples and aspects of the invention. It should be appreciated that the scope of the invention includes other embodiments not discussed in detail. Various other modifications, changes and variations which will be apparent to those skilled in the art may be made in the arrangement, operation and details of the method and apparatus of the present invention disclosed herein without departing from the spirit and scope of the invention as described here.

The present application discloses a device for safely accessing bone marrow and other tissues. While the embodiments disclosed herein are described mostly with reference to a bone marrow access device, it is understood that the teachings apply to other embodiments and devices for accessing tissue regions other than bone marrow. For example, the accessed tissue could be any other tissue or a cavity within any organ or body part, such as the sinus cavity, the uterus, the stomach, the spine, etc.

The bone marrow access device allows the user a precise and controlled method to penetrate a needle into a bone marrow cavity without the risk of puncturing through the entire bone and risking penetration of tissue or organs posterior to the bone. Consequently, this device enables the user to safely aspirate bone marrow from high risk and thin profile puncture sites such as the sternum. Rather than relying on the user's judgment to determine penetration depth, the device automatically prevents the user from pushing the needle past the bone marrow tissue. The same access device can also be used for infusing drugs or medication into the bone marrow cavity.

The present embodiments comprise a bone penetrable aspiration needle and an actuator configured to engage with the needle. The actuator, which may be a rotatable handle or knob, is used for advancing the needle through the bone wall and into a bone marrow cavity. The actuator engages with the needle via a mechanical sensor assembly.

The mechanical sensor assembly is configured to initially engage the actuator with the needle, thereby allowing the actuator to advance the needle through the bone wall. Once the needle passes through the bone wall and reaches the bone marrow cavity, the mechanical sensor assembly automatically disengages the actuator from the needle, preventing the needle from advancing any further into the bone marrow cavity.

In order to sense entry of the aspiration needle into a bone marrow cavity, the mechanical sensor assembly comprises a spring-loaded pin which extends through the needle. When the needle punctures through the bone wall, the distal tip of the spring-loaded pin meets with reduced resistance and extends beyond the distal tip of the aspiration needle and into the bone marrow cavity. The sensor assembly is configured to sense this extension and automatically trigger the disengagement of the actuator from the needle.

The mechanical sensor assembly thus senses the needle's entry into the bone marrow cavity without reliance on the user's judgment of penetration depth, thereby providing precise needle penetration with minimized risk of puncturing through the entire bone. This allows safe bone marrow aspiration from high risk and thin profile puncture sites such as the sternum.

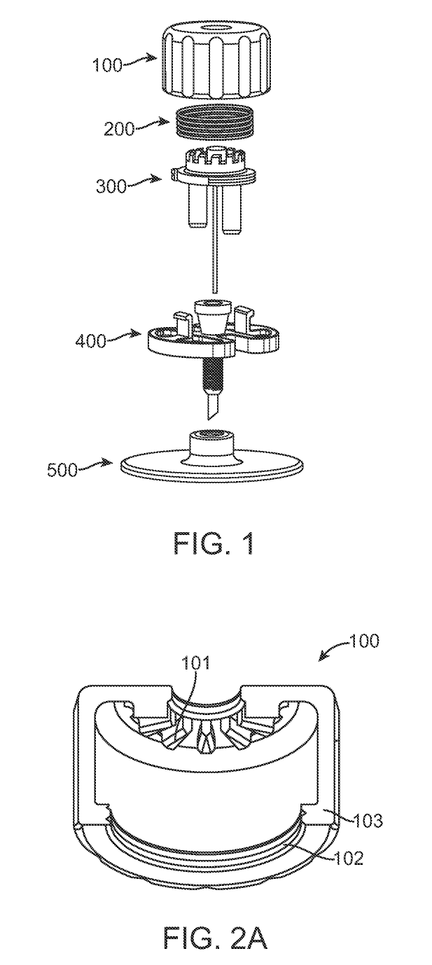

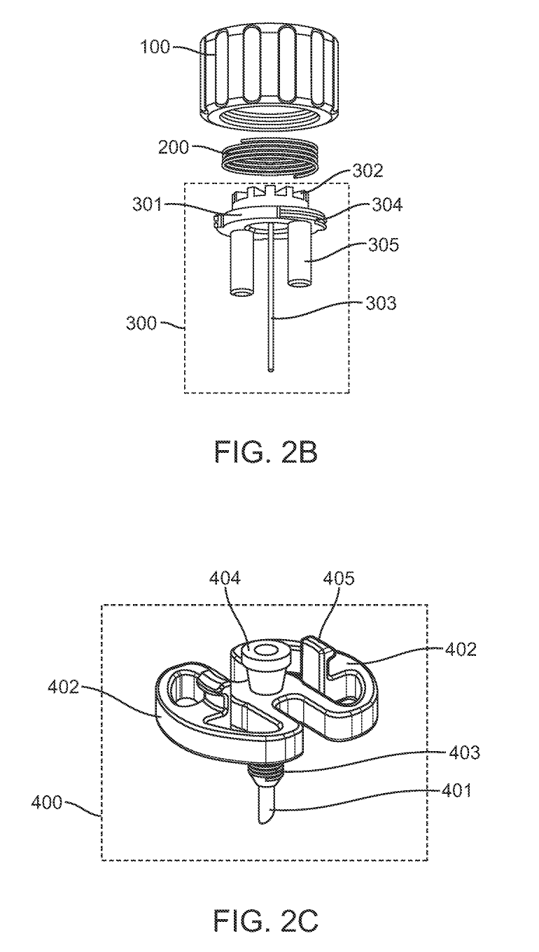

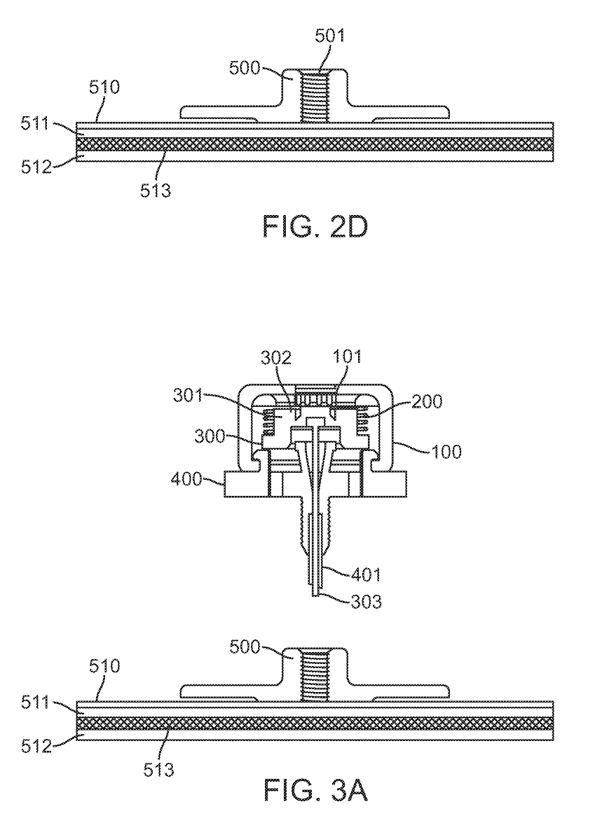

An exemplary BMA device in accordance with an embodiment of the present invention is shown in FIG. 1. Such a device comprises an actuator 100, a spring 200, a pin assembly 300, a needle assembly 400, and an insertion base 500 (hereinafter also referred to as base 500). An oblique sectional view of actuator 100 is shown in FIG. 2A. Actuator 100 is exemplarily a rotatable knob comprising teeth 101 to engage corresponding teeth in pin assembly 300. Optionally, some portion of the actuator 100 knob comprises an internal threaded portion 102 within a recess 103, for engaging the pin assembly 300.

The pin assembly 300, as shown in FIG. 2B, comprises a pin retainer 301 with teeth 302 formed on the proximal portion to engage the teeth 101 (not shown in this view) of actuator 100. The pin 303 is affixed to the pin retainer 301 and extends in the distal direction. The pin retainer 301 optionally has an externally threaded portion 304 to allow insertion of the pin retainer 301 into the actuator 100 in such a manner that the pin retainer 301 is locked within the actuator 100 chamber but remains free to rotate inside the actuator 100. Optionally, two aligning pins 305 are located below the threaded portion 304 to aid the transfer of torque from the pin assembly 300 to the needle assembly 400.

The needle assembly 400, as shown in FIG. 2C, comprises unlatching tabs 402 at one end and an aspiration (or infusion) needle 401 with a bone penetrable point at the other end. This bone penetrable aspiration needle 401, hereinafter also referred to as needle 401, comprises a bore to allow for aspiration or infusion. The needle 401 with the bone penetrable point can be single faceted, multi-faceted, or beveled, and may include designs such as, but not limited to: multi-faceted diamond design, hypo point design, back bevel design, trocar design (or variations such as stepped trocar design), conical point design (or variations such as truncated conical point design), barrel tip design, barb point design, lancet point design, franseen point design, arrowhead point design, or other such designs or combinations thereof. Further, the term needle is used to also represent an access or delivery port.

The needle assembly 400 optionally comprises an externally threaded portion 403 extending along some portion of the length of the needle to engage with the threaded portion of the base 500. At the top end of the needle 401 is located a fitting 404, for example a Luer fitting, to receive the pin 303. Needle assembly 400 additionally comprises fastening elements, such as needle assembly latches 405, which enable the needle assembly 400 to engage the actuator 100.

An exemplary insertion base 500, as shown in FIG. 2D, comprises a substantially flat distal end and is configured to be placed on the skin and optionally affixed to the skin using an adhesive or any other affixing or anchoring means. Optionally, base 500 has internally threaded portion 501 to receive the corresponding threaded portion of needle 401. Also shown are skin, subcutaneous tissue, and periosteum, collectively labeled as 510, the anterior bone cortex 511, posterior bone cortex 512, and bone marrow cavity 513.

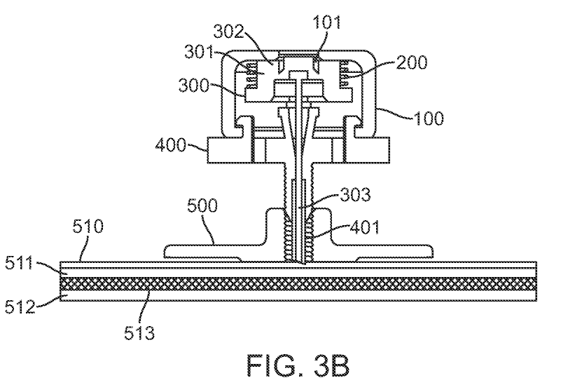

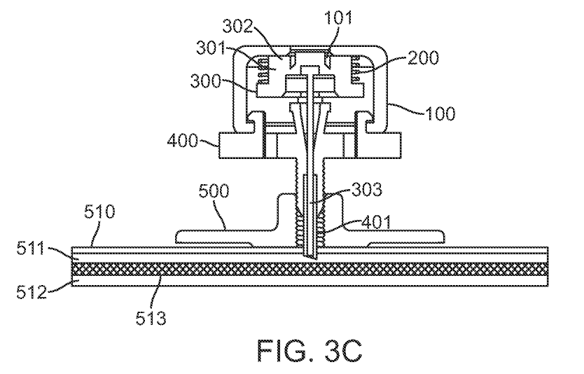

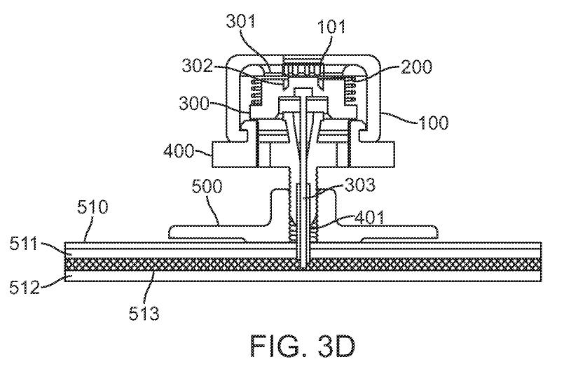

FIGS. 3A through 3D describe the operation of the bone marrow access device. The various parts heretofore described are assembled in the order shown in FIG. 1 and the assembled device is illustrated in FIG. 3A. Actuator 100 comprises the spring 200, which in turn engages the pin assembly 300, which is mechanically engageable with needle assembly 400. Pin 303 extends beyond the point of the needle 401. Simultaneously, spring 200 is fully extended prior to insertion into the insertion base 500. In this initial position, teeth 101 of the actuator 100 are disengaged from the corresponding teeth 302 of the pin assembly 300. Consequently, rotation of the actuator 100 knob does not transfer to the pin assembly 300 and needle assembly 400.

The device is shown positioned to penetrate the anterior bone cortex 511 in FIG. 3B. The needle 401 is inserted into the base plate and advanced by rotating the actuator 100 such that threaded portion 403 is advanced through threaded portion 501 of the base 500, until the tip of the needle 401 comes into contact with the anterior bone cortex 511. As the needle 401 advances towards the bone cortex 511, pin 303 reaches the bone cortex 511 before needle 401 does. From that point on, further advancement of the needle 401 towards the bone cortex 511 causes pin 303 to be pushed into the needle 401, due to resistance from the anterior bone cortex 511. This causes the pin assembly to move in a proximal direction relative to and towards the actuator 100, enabling teeth 302 to engage with corresponding teeth 101 of actuator 100 and thereby engaging the actuator 100 with the needle assembly 400.

Once the actuator 100 has been engaged with the needle assembly 400, further advancement of the needle 401 through the bone is accomplished by continuing to rotate actuator 100, as shown in FIG. 3C.

The device having penetrated the bone marrow is shown in FIG. 3D. In this position, pin 303 no longer encounters high resistance, because it has entered the soft bone marrow. The spring 200 thereby also no longer encounters high resistance and thus decompresses. The decompression of spring 200 causes the pin 303 to move out of the point of the needle 401 and advance further into the bone marrow cavity. Because of this movement of the pin assembly 300 distally and away from the actuator 100, teeth 302 of the pin assembly 300 disengage from corresponding teeth 101 of the actuator 100, thereby disengaging the pin assembly 300 from the actuator 100. Consequently, further rotation of the actuator 100 does not advance the needle 401, thereby preventing further penetration of needle 401 through the bone marrow and into the posterior bone cortex 512.

As described above, the pin assembly 300 operates based on a resistance differential between the bone cortex and the softer bone marrow. Accordingly, the pin assembly 300, and in particular the tension of the spring 200, may be adjusted or tuned in order to configure the device for use at the desired bodily site, based on the resistance differential present or likely to be encountered at the desired site. Such resistance differentials are calculated based on resistance values of the desired tissue as known in the art.

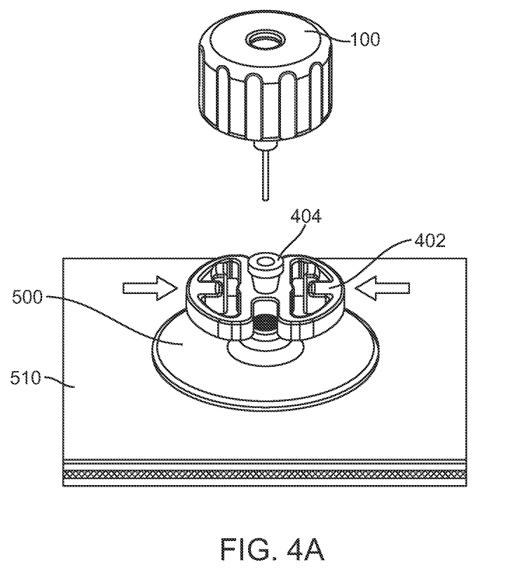

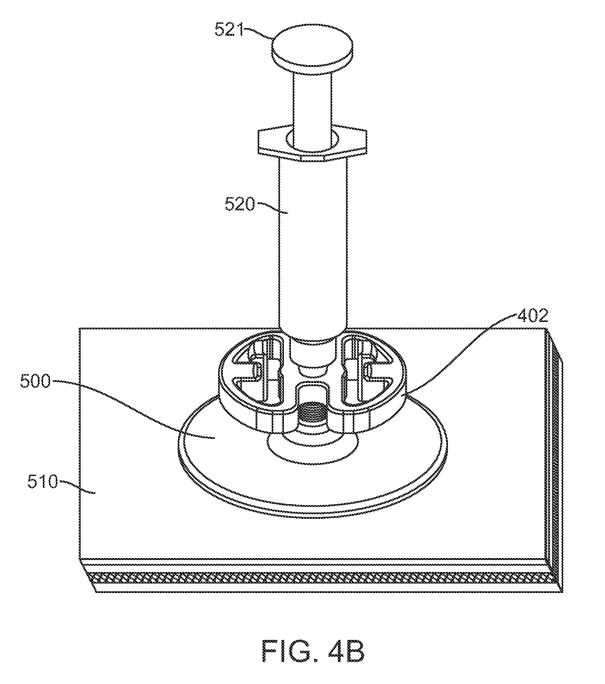

As shown in FIG. 4A, once the needle 401 has entered the bone marrow, the actuator 100 and pin assembly 300 (not shown in this view) are disengaged from the needle assembly 400 by the user releasing the unlatching tabs 402, for example by pressing or squeezing in the direction indicated by the arrows. Luer fitting 404 is thus left open to the bone marrow for access by an extraneous device such as a syringe, an infusion tube, or other suitable device.

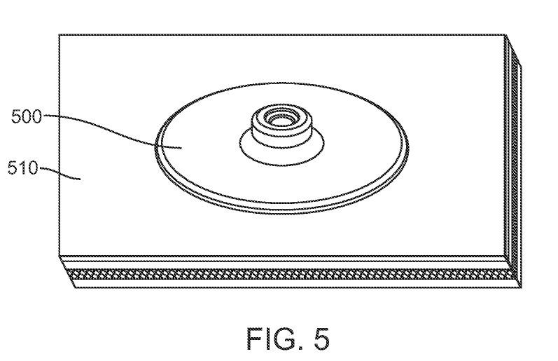

FIG. 4B shows a syringe 520 used to access the bone marrow. The syringe 520 is shown comprising a plunger 521 to facilitate extraction on injection. The syringe 520 can be used to extract bone marrow, or to introduce drugs or other substances into the bone marrow cavity. After access is complete, the needle assembly 400 is removed by unscrewing the needle assembly 400, leaving the insertion base 500 behind, as shown in FIG. 5. The insertion base 500 is then removed, or optionally left in place for any subsequent procedures that might utilize the insertion base 500.

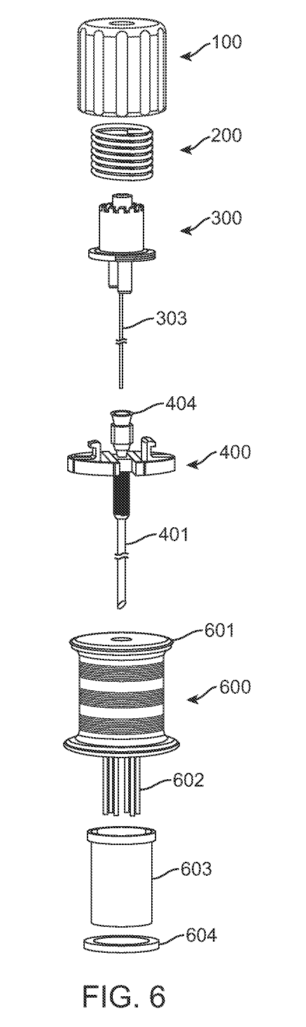

Another embodiment of the bone marrow access device is shown in FIG. 6. This embodiment addresses the challenges posed by varying skin and subcutaneous tissue thicknesses. As can be understood, the thickness of the subcutaneous tissue varies enormously. For example, this variation could be in the range of 1 mm (pediatric cases) to 4 cm or more (obese patients). The embodiments disclosed below take into account the various skin thicknesses that may be encountered and use a modified base to accommodate such varying skin thicknesses.

As shown in FIG. 6, this embodiment comprises the actuator 100, spring 200, pin assembly 300, pin 303, needle assembly 400, and needle 401, as described in the first embodiment above. In lieu of base 500 of the first embodiment, the embodiment of FIG. 6 comprises a base 600 configured to be held easily by the user. The pin 303 and needle 401 can be of variable length to reach bone locations in varying depths of soft tissue.

The base 600 comprises a receiving end 601 for receiving the needle 401. The base 600 additionally comprises a plurality of needles 602 configured to anchor or stabilize the device at the access site; for example, within the subcutaneous tissue and some portion of the bone cortex, such as the anterior surface. Simultaneously, the needles 602 may aid the user to reference the bone. The length of the needles 602 is comparable to the length of the needle 401. Optionally, the needles 602 are spring-loaded to move within the base 600. This embodiment additionally or optionally comprises a needle protector 603 which is configured to slide into the base 601 upon insertion of needles 602 into the skin. A base ring 604 is optionally provided to aid in positioning, stabilizing, or anchoring the device.

The base 600 is placed onto the skin and pressure is applied to it such that needles 602 penetrate the tissue. In this position, the needle protector 603 covers the needles 602. Actuator 100 comprises the spring 200, which in turn engages the pin assembly 300, which is mechanically engaged with needle assembly 400, thereby penetrating the bone tissue, while pin 303 is kept pressed within the needle 401. Needles 602 advance to a variable depth depending on the thickness of the tissue overlying the bone. Pressure is continuously applied to base 600 until the needles 602 encounter bone. When they reach the bone, advancement of the needles 602 stops due to the resistance provided by the bone. The needles 602 are configured to penetrate the soft tissue in a manner sufficient to provide additional anchorage at the access site, thereby facilitating advancement of the needle 401. Further, when the needles 602 have reached the surface of the bone, the distal tip of needle 401 will have reached the anterior surface of the bone cortex also. Similar to that described in FIGS. 3A through 3D, when the needle 401 is against the bone, pin 303 is pushed into the needle 401 due to resistance from the anterior bone cortex. This causes the pin assembly 300 to move upwards, enabling teeth 302 to engage with corresponding teeth 101 of actuator. Further advancement of the needle through the bone is accomplished using the actuator 100 knob.

Similar to FIG. 3D, when the needle 401 passes the bone cortex and into the bone marrow, the pin 303 encounters reduced resistance. The spring 200 thereby also encounters reduced resistance and thus decompresses. The decompression of spring 200 causes the pin 303 to move out of the point of the needle 401 and further enter the bone marrow cavity. Because of the downward movement of the pin assembly 300, teeth 302 disengage from corresponding teeth 101 of the actuator 100 and, consequently, further rotation and advancement of the needle 401 using actuator 100 is not possible. This prevents penetration into the posterior bone cortex. Thereafter, similar to that shown in FIG. 4A, the actuator 100 and pin assembly 300 are removed from the needle assembly 400, allowing access to the bone marrow through fitting 404.

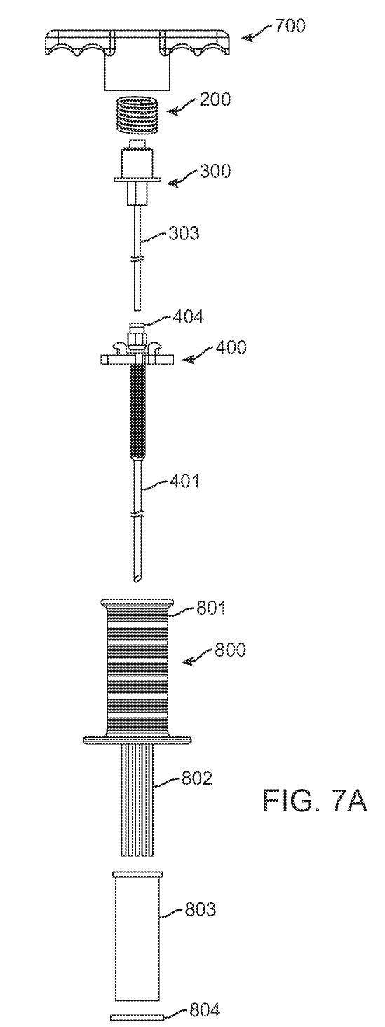

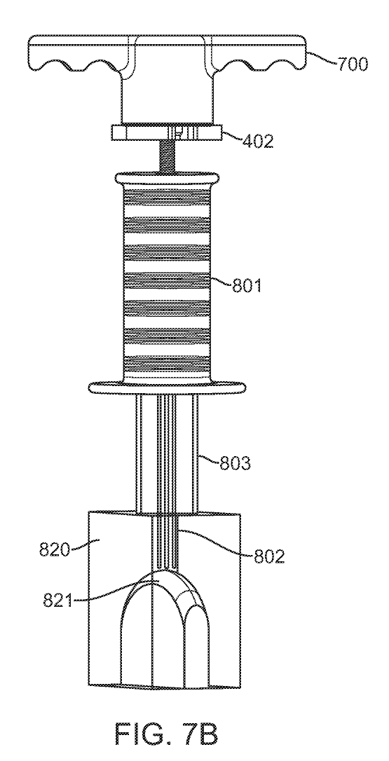

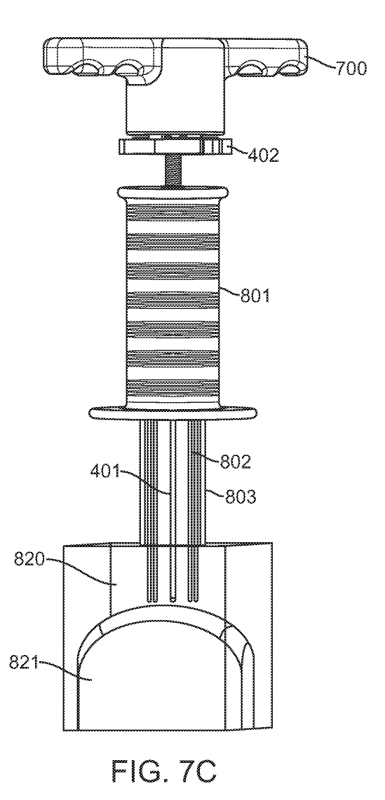

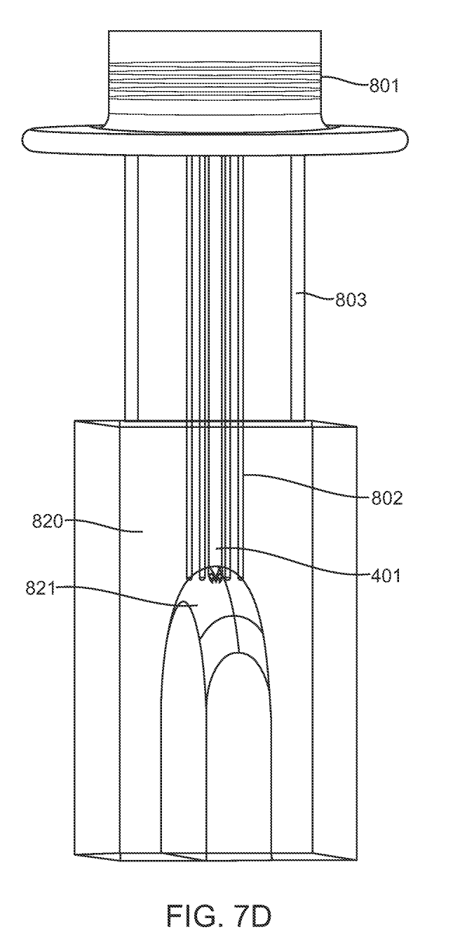

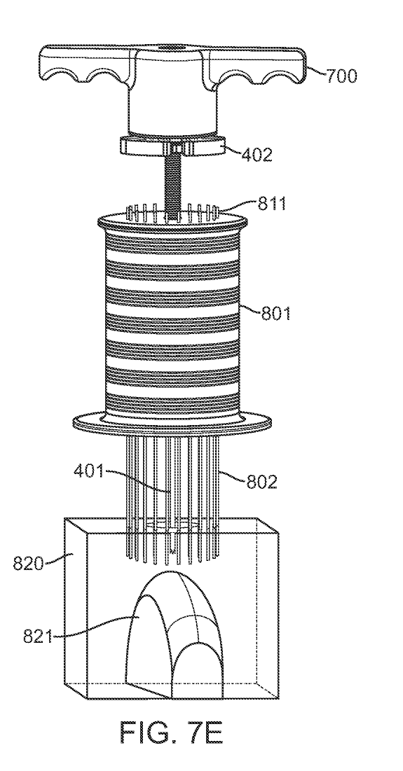

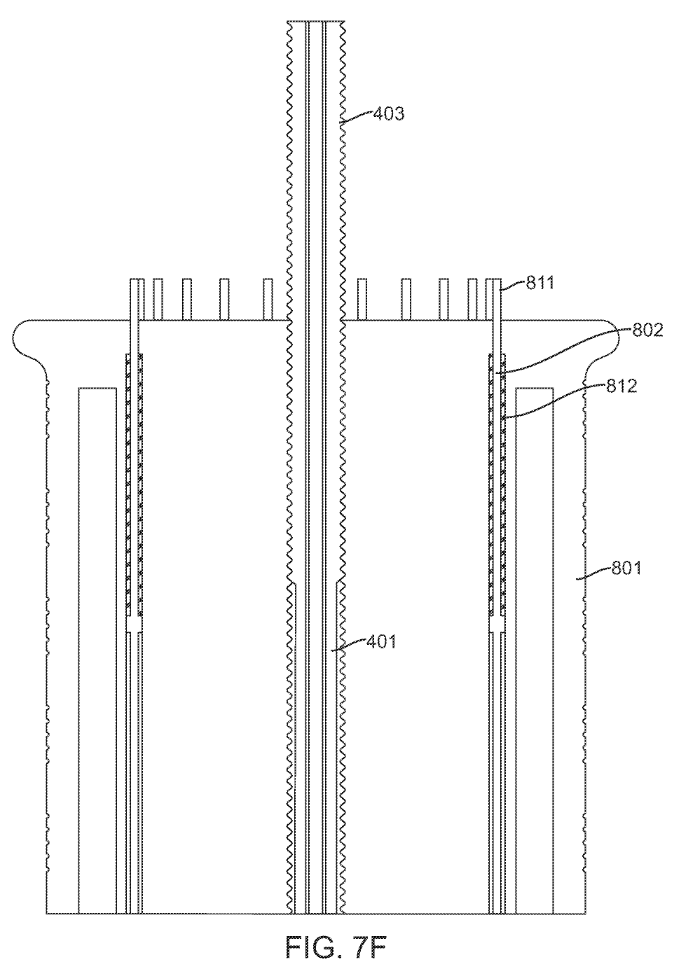

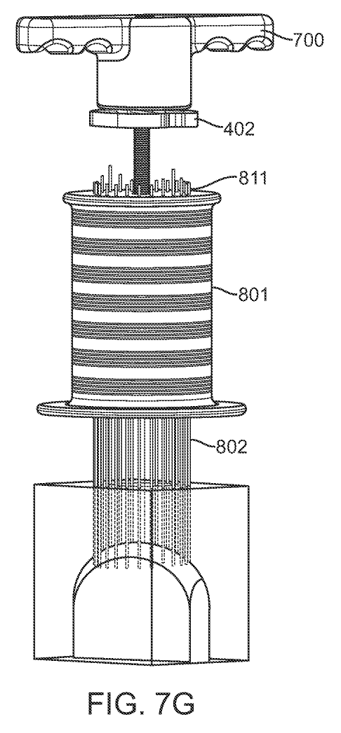

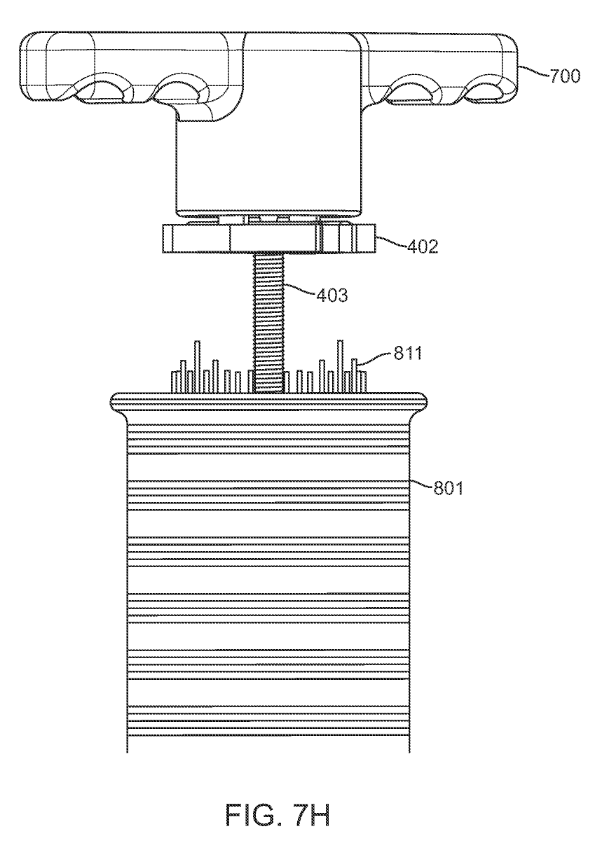

A third embodiment of the present invention, as shown in FIGS. 7A through 7G, is configured to access deeper bone marrow sources, for example the iliac region of the pelvis. As shown in FIG. 7A, in this embodiment, actuator 700 is optionally configured to provide a full-handed grip and additional rotational leverage. This embodiment further comprises a pin 303 and needle 401 configured to penetrate the deeper tissue of the iliac region, and a base 800. The base 800 comprises a receiving end 801 for receiving the needle 401. The base 800 additionally comprises a plurality of needles 802 configured to anchorably enter the skin. The lengths of the needles 802 are comparable with the length of the needle 401. This embodiment additionally or optionally comprises a needle protector 803 which is configured to slide within the base 800 upon insertion of needles 802 into the skin. A base ring 804 is optionally provided to aid in positioning, stabilizing, or anchoring the device. Similar to the previous embodiments, actuator 100 comprises the spring 200, which in turn engages the pin assembly 300, which is mechanically engaged with needle assembly 400. Pin 303 initially extends beyond the point of the needle 401. This embodiment differs from that shown in FIG. 6 in that the diameter of the concentricity of the needles 802 is smaller so as to correspond with at least some portion of the iliac crest. Additionally, the number of needles 802 in this embodiment could comprise any number sufficient to reference the iliac crest.

Additional details of the third embodiment of the device are shown in FIGS. 7B through 7G. Pressure is applied to the base to push the needles 802 into the soft tissue 820 overlaying the iliac crest 821. FIG. 7B shows the device with needles 802 that have penetrated the tissue 820 above the iliac crest 821. FIGS. 7C and 7D shows an enlarged view of the needles 802 at the point where they have penetrated soft tissue 820 and have reached the iliac crest 821. FIG. 7C shows that as the needle 401 references the iliac crest, pin 303 has retracted within the needle 401. When it reaches the iliac crest, as shown in FIG. 7D, needle 401 is flush against the iliac crest 821.

Additionally or alternatively, as shown in FIG. 7E, the needles 802 are vertically movable within the base such that the proximal points 811 of the needles 802 may project out of the top of the base 800. This aids the concentric needles to correspond with and stabilize upon the surface of the iliac crest, thereby aiding the needle 401 to stabilize upon any curved or irregular surface.

Optionally, needles 802 are coupled to spring-based mechanisms, as shown in FIG. 7F, which shows the base 800 in cross section. The needles 802 are configured to be kept in position by the action of the springs 812. When pressure is applied to the base 800, the needles 802 are thereby allowed to penetrate tissue 820. When the needles 802 encounter resistance as they encounter bone, the pressure acts against the springs 812 to move the needles vertically to enable them to adjust to the contour of the curved or irregular surface of the iliac.

FIG. 7G illustrates the device with the needles 802 anchored against the iliac crest 821. Needles 802 adjust to the contour of the top surface of the iliac crest. Correspondingly, the contour taken up by the needles is observed through the profile of the projecting ends 811 visible on top of the base 800. A magnified view of the projecting needles is shown in FIG. 7H, where the projections 811 are shown assuming the contour of the iliac crest against which the needles 802 have been anchored. The distribution of the projecting ends 811 indicates whether the device is centered or otherwise properly positioned for bone marrow access with reference to the surface of the iliac crest 821. The particular example distribution of the needles shown in FIGS. 7G and 7H indicates that the device is centered with respect to the iliac crest. Optionally, the device comprises a locking mechanism (not shown) to lock the needles 802 in place after the device has been determined to be properly positioned, thereby inhibiting movement of the device with respect to the bone. The ability of the surgeon to accurately place the device perpendicular to the surface of the bone enhances the safety and reduces time taken to complete the procedure.

After the base is anchored as described in FIGS. 7A through 7G, the needle 401 is advanced by rotating the actuator 100 similar to that described in FIGS. 3A through 3D. Similar to that described in FIGS. 3A through 3D, when the needle 401 is against the bone, pin 303 is pushed into the needle 401 due to resistance from the anterior bone cortex. This causes the pin assembly 300 to move upwards, enabling teeth 302 to engage with corresponding teeth 101 of actuator 100. Further advancement of the needle 401 through the bone is accomplished using actuator 100 knob.

Similar to FIG. 3D, when the needle 401 passes the bone into the bone marrow, the pin 303 no longer encounters resistance. The spring 200 thereby also encounters no resistance and thus decompresses. The decompression of spring 200 causes the pin 303 to extend beyond the point of the needle 401 and further enter the bone marrow cavity. Because of the downward movement of the pin assembly 300, teeth 302 disengage from corresponding teeth 101 of the actuator 100 and, consequently, further rotation and advancement of the needle 401 using actuator 100 is not possible. This prevents penetration into the posterior bone cortex. Thereafter, similar to that shown in FIG. 4A, the actuator 100 and pin assembly 300 are removed from the needle assembly 400, allowing access to the bone marrow through fitting 404.

While the above embodiments provide access to tissue from parts located close to the skin or those that are accessible through the external surface of the body by piercing the skin, it is also desirable to provide safe access to tissue or cavities in internal body parts that may be more readily approached through body passageways such as the digestive or respiratory systems of the body. Further embodiments disclose such a device in which a flexible catheter is used for such internal access. In these embodiments, the needle is connected to the needle assembly through a flexible coil, while the pin is attached to the pin assembly via a flexible shaft. The needle is sheathed in a flexible catheter. The device is used to access tissue by operating the actuator at the proximal end to advance the needle through tissue, as in the previous embodiments.

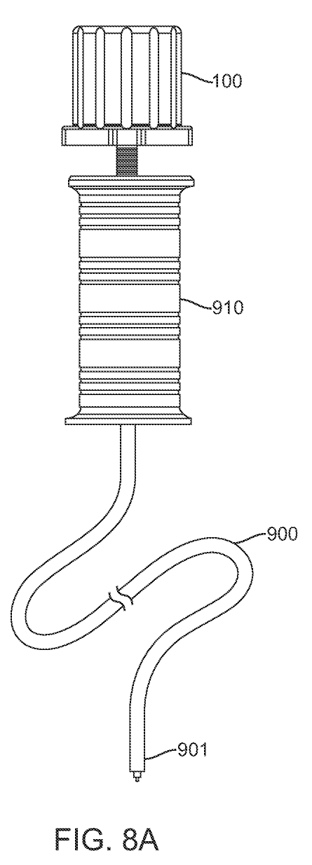

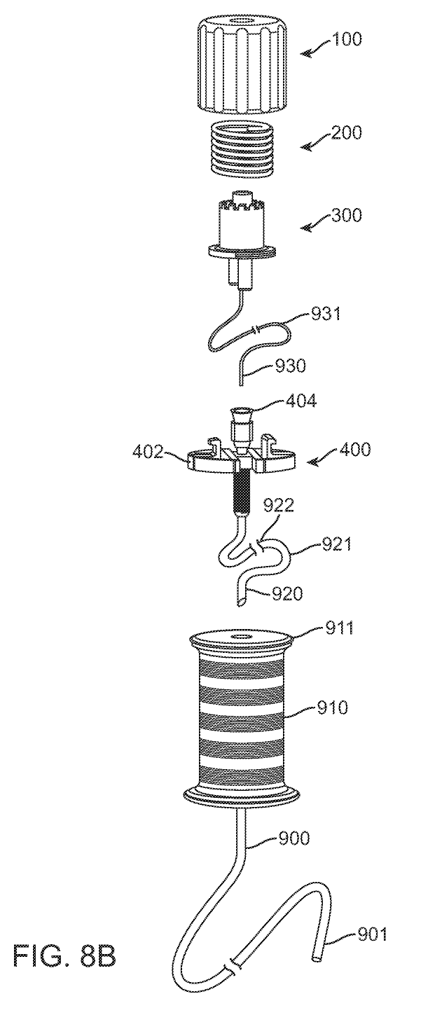

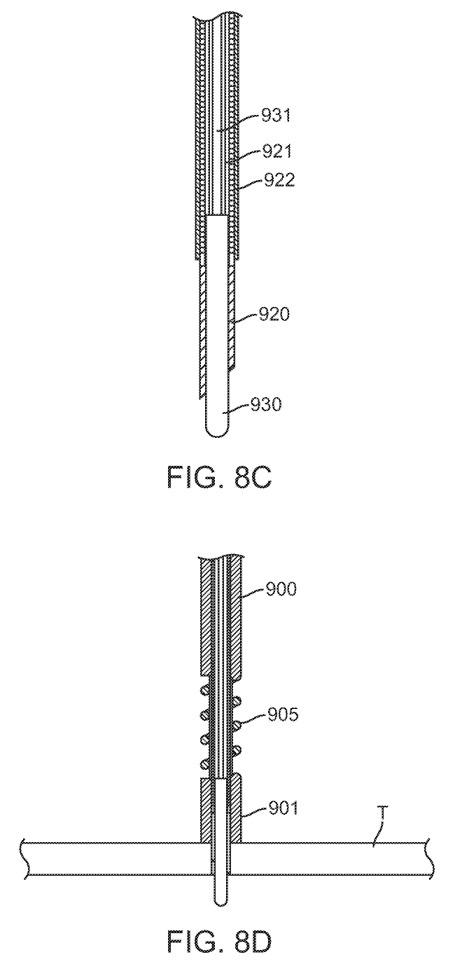

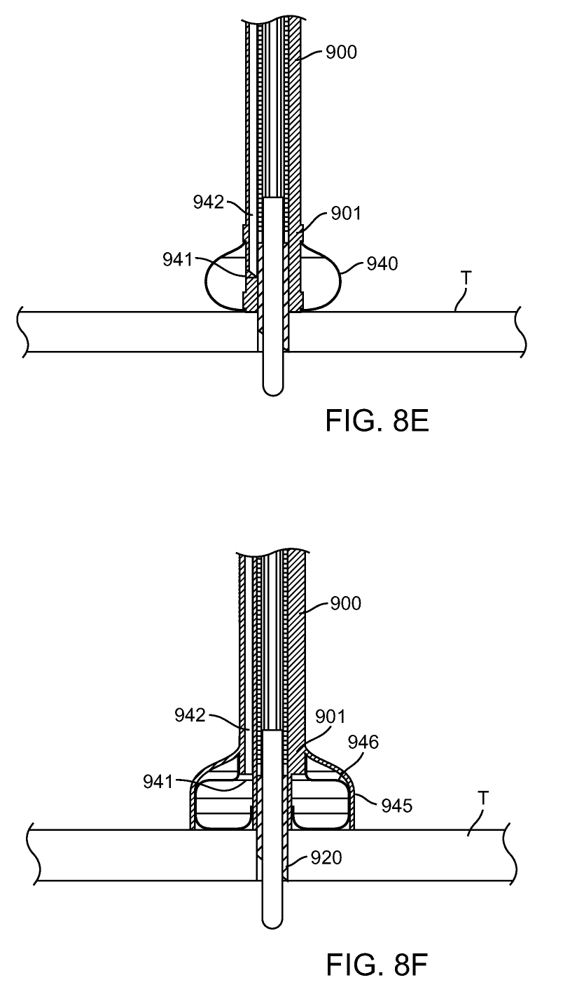

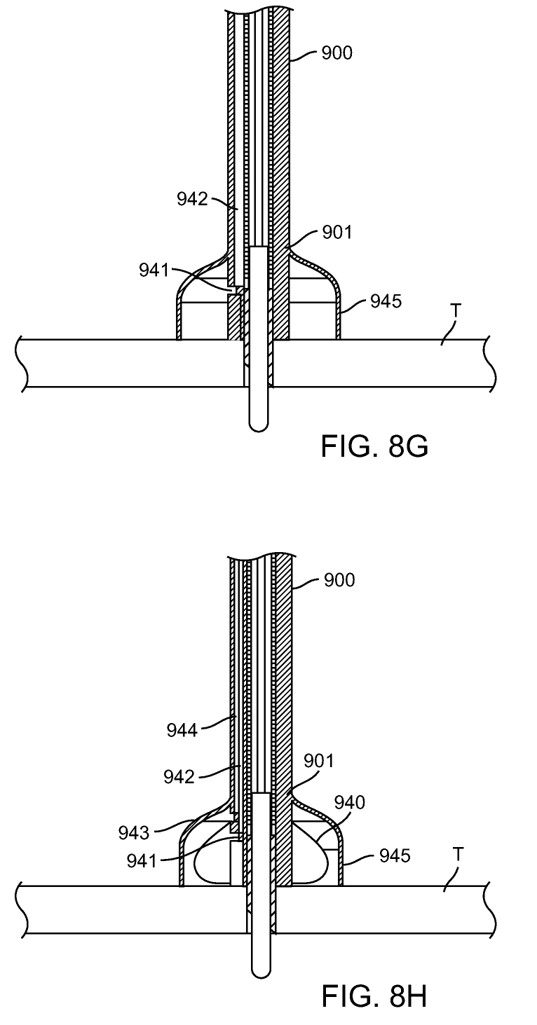

Various embodiments of the catheter-based device of the present invention are shown in FIGS. 8A through 8H and are described further with reference to these figures. As shown in FIG. 8A, a flexible catheter 900 is attached to an elongated base 910 and comprises a distal tip 901. Actuator 100 is attached to the top of the base 910, as in the previous embodiments. Further details of the device of this embodiment are shown in FIGS. 8B through 8D. FIG. 8B shows an exploded view of the device with its component parts while FIGS. 8C and 8D show the needle and pin in greater detail. Starting with FIG. 8B, a needle 920 is attached to a needle assembly 400 via a tubing 921 reinforced with a flexible coil 922. Flexible coil 922 is configured to exhibit flexibility and torque transfer, while maintaining sufficient rigidity in the longitudinal direction to drive needle 920 into tissue. A pin 930 is connected to a pin assembly 300 through a flexible shaft 931. As shown in FIG. 8C, needle 920 is sheathed in tubing 921 and is coupled to the distal end of flexible coil 922. Reinforced tubing 921 acts as a lumen through which to access tissue and to allow movement of pin 930. Pin 930 is attached to the distal end of flexible shaft 931 and projects from the distal tip 901 of the catheter 900 (as shown in FIG. 8D).

Catheter 900 of the device in the assembled condition as shown in FIG. 8A is inserted into any body passageway to access tissue such as an internal organ or body part. Tip 901 of catheter 900 is pressed against the internal body surface to be penetrated, which presses the projecting portion of pin 930 into the catheter 900, as shown in FIG. 8B. This movement is transferred through flexible shaft 931 to the pin assembly 300, thereby engaging one or more teeth on top of the pin assembly 300 with corresponding teeth on the actuator 100. Turning actuator 100 rotates needle assembly 400 so that the threaded portion thereof engages with internal threads of the elongated base 910, thereby advancing reinforced tubing 921. The axial and rotating motions of the actuator 100 are transferred to needle 920 through flexible coil 922. Needle 920 is thereby advanced through tissue using actuator 100 until more compliant tissue, or an internal body cavity, is encountered, which results in projection of the pin 930 beyond the tip of the needle 920. At this point, pin assembly 300 disengages from the actuator 100 under the action of spring 200 and further advance of needle 920 is stopped.

After the needle penetrates to the location where tissue or a cavity is to be accessed, the actuator 100 is detached from the needle assembly by pressing unlatching tabs 402 (FIG. 8B), exposing a luer lock fitting 404. A syringe can be connected to the luer lock fitting 404 to aspirate a tissue sample or to infuse a drug, as may be required. Additionally and optionally, the opening created using the catheter device can be used as access port for inserting additional diagnostic or therapeutic devices into the accessed cavity.

The body of catheter 900 is made of biocompatible flexible polymeric material as is well known in the art. However, to reduce the possibility of the distal tip 901 of catheter 900 penetrating internal body surfaces, a force-limiting spring is included as part of the catheter in another embodiment shown in FIG. 8D. As shown in FIG. 8D, distal tip 901 of catheter 900 is connected to the rest of the catheter 900 by a spring 905. When placed against an internal body surface T, spring 905 compresses, limiting the force on the tissue surface T. Needle 920 and pin 930 are then carefully advanced to penetrate tissue and access a cavity or tissue therein as required.

In another embodiment shown in FIG. 8E, the distal tip 901 of the catheter terminates in an inflatable balloon, reducing concentration of force and lowering the risk of inadvertent penetration by the tip of the catheter. This embodiment allows better referencing of the tissue boundary by the catheter tip. As shown in FIG. 8E, a balloon-tipped catheter is moved into position within the body. Balloon 940 in initially in a deflated configuration. Balloon 940 is inflated when required, using an inflation port 941 supplied by an inflation lumen 942. Alternatively, the catheter can also be advanced with balloon 940 in an inflated configuration.

In another embodiment, a cup made of relatively non-flexible material is attached to the tip 901 of the catheter, as shown in FIG. 8F. Cup 945 is made of relatively non-flexible material and is used to shape inflation of balloon 946 through inflation port 941 via inflation lumen 942. Catheter 900 is moved into position onto the internal body surface T for tissue access so that cup 945 is placed in contact with the surface. Inflating the balloon 946 forces tissue surface T to conform to the balloon surface so that substantially perpendicular contact with tissue can be provided for needle 920.

In another embodiment shown in FIG. 8G, the relatively non-flexible cup acts as a vacuum anchor. Cup 945 is open to suction port 941 through a suction lumen 942. In operation, the tip 901 of catheter 900 is placed against the tissue surface T and vacuum is applied through the suction port 941 to provide substantially perpendicular contact of the tissue surface T with the tip 901 of the catheter 900.

In another embodiment shown in FIG. 8H, the vacuum anchor could additionally have a balloon 940 attached to the tip of the catheter 901, wherein the balloon does not entirely fill the vacuum cup 945, but leaves an annular portion available for vacuum application. The catheter can be used with the balloon 940 in the inflated condition. Vacuum is applied through suction port 943 (attached to suction lumen 944) to provide conformity of the catheter tip to the tissue. The balloon 940 provides support for the tissue surface being accessed, while anchorage is provided by vacuum suction. The device of this embodiment reduces tissue trauma while accessing delicate or soft body parts. Similar to the embodiment shown in FIG. 8G, the cup 945 is open to suction port 941 through suction lumen 942.

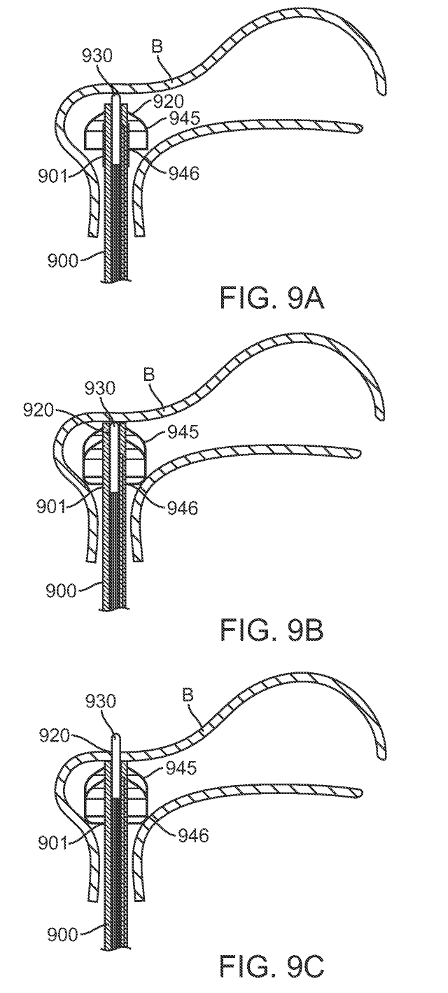

In another embodiment of the device shown in FIGS. 9A-9C, the device comprises a balloon placed near the tip that enables anchoring within a body cavity, such as the intranasal space for access to the sinuses. As shown in FIG. 9A, the distal end 901 of the catheter 900 comprises a balloon 946 placed within a cup of relatively non-distensible or non-compliant material 945 that is designed to direct the inflation of the balloon backwards towards the proximal end of the catheter. FIG. 9A shows the catheter placed in position within the intranasal space for accessing a sinus cavity. When inflated as shown in FIG. 9B, the balloon 946 expands backwards and pushes the tip of the catheter perpendicularly against the bone B by referencing the walls of the intranasal space so that the pin 930 is pushed into the device and engages the actuator 100 (not shown in this figure). With the catheter so stabilized in position, the actuator is used to advance needle 920 into the bone B for accessing the sinus cavity, as shown in FIG. 9C. Further advance of the needle is prevented when pin 930 penetrates into the cavity, thereby providing for safe tissue access, as in previous embodiments.

Although the device has been shown as operable in relation to the nasal cavity and sinuses, it can be used in any other cavity or space within the body where a similar anchoring is possible.

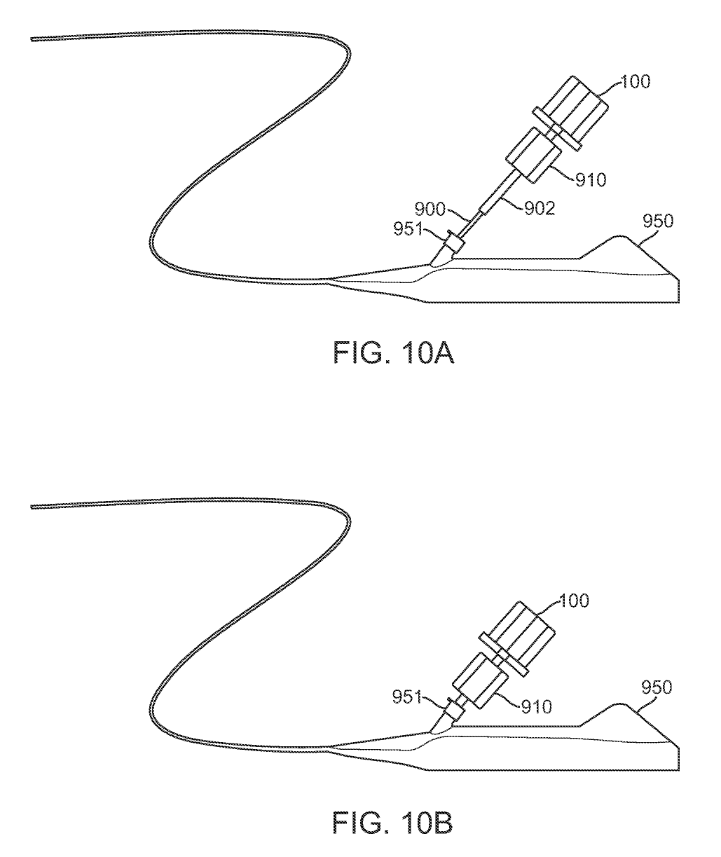

The catheter device of the present invention can be used through a second access device. This access device can be a laparoscopic trocar or a viewing scope, such as a bronchoscope, as shown in FIGS. 10A and 10B. The catheter 900, which is associated with the base 910 (which is in turn associated with the actuator 100), is inserted through the valve port 951 into the working channel of a viewing scope 950 as shown in FIG. 10A. The proximal portion 902 of the catheter 900 is sized to a diameter suitable for locking the catheter 900 within the lumen of the viewing scope 950 at a desired position using valve port 951. The catheter 900 of the present invention is shown locked in position to a viewing scope 950 in FIG. 10B.

The catheter device of the present invention is particularly suitable for accessing tissue of internal body parts such as the sinuses, stomach, lung, ear drum, uterus, kidney, gall bladder and other organs covered by hard or soft tissue.

In the above embodiments, the needle, the coil lumen, the flexible shaft and the pin are made of a suitable bio-compatible material such as surgical stainless steel. Other components are constructed of suitable biocompatible materials such as polypropylene, polycarbonate, PTFE, or PEBAX.

The devices, as described above, are driven by rotation of a rotatable actuator. Alternatively, such rotation could be provided by spring force, vacuum pressure, air pressure, shape-memory alloys (e.g., Nitinol, etc.), Nitinol actuator wires (e.g., Flexinol, etc.), or other mechanisms. Alternatively, such rotation could be provided electrically, for example through motor controlled devices, solenoid-actuated, or piezo-actuated devices.

It is further contemplated that the tissue access devices described herein may also be used as a trocar through which an instrument or device may be introduced into the second tissue region. Such an instrument may be used to collect further diagnostic information, and examples of such instruments include, but are not limited to: an optical scope or catheter, an ultrasound probe or catheter, a Doppler probe, a thermocouple, a pH sensor, etc. Alternatively or in combination, therapeutic devices may be introduced thusly into the second tissue region in order to provide further treatment, examples of which include, but are not limited to: a radio-frequency (RF) ablation device, a cryogenic catheter, etc.

Further, alternative to the mechanical sensor disclosed above, other sensing mechanism can be used to detect the bone marrow cavity. For example, a pH sensor can be incorporated into the end of the pin to detect changes in pH when the bone marrow cavity is accessed.

Alternatively, the sensor can be an optical detector that detects color and can be incorporated into the tip of the needle to detect the change in color while penetrating the bone marrow cavity.

Alternatively, a temperature sensor placed at the tip of the needle or at the tip of the pin could sense a sudden temperature increase upon penetrating the bone marrow cavity.

In yet another embodiment, advancement of the needle into the bone marrow cavity could be detected by air pressure variation. In such an embodiment, the needle allows for detection of changes in air pressure, and a sudden change (such as a sudden drop) in air pressure upon accessing the cavity is sensed and advancement is stopped. Optionally, the needle may carry pressurized air in order to provide a more easily detectable amount of change in air pressure.

Alternatively, the pin and the needle are electrically isolated from each other. As the needle advances through bone, a certain capacitance value is registered between the two members. On reaching the bone cavity, the capacitance drops, and such drop is signaled to the device which is configured to stop needle advancement upon receipt of such signal.

In another variation of the device, a moisture sensor is placed at the tip of the needle or the pin. The needle and pin initially encounter tissue with high moisture content before the bone is penetrated. Thereafter, the moisture content in the bone would register a low value. On reaching the bone marrow cavity, the sudden increase in moisture content is sensed and is signaled to the device which is configured to stop needle advancement upon receipt of such signal.

Alternatively, an electrochemical sensor can be used. Electrochemical potential across bone is negligible, due to the bone's insulating nature. On penetrating to the bone marrow cavity, a higher potential is encountered between a reference electrode and one placed in the cavity. This is detected and is signaled to the device which is configured to stop needle advancement upon receipt of such signal.

Alternatively, acoustic waves can be conveyed through the pin. When bone is being penetrated, an echo signal is reflected from the internal surface of the bone, which is detected. On penetrating through to the bone marrow cavity, the acoustic reflections attenuate, leading to a drop in the reflected signal, which is used to sense penetration and stop advancement. Electrical impedance of bone is high due to its insulating nature. When the device penetrates into the marrow cavity, the moist tissue located therein causes a sudden drop in impedance. This drop is sensed and the device is configured to stop needle advancement upon receipt of such signal.

Additionally or alternatively, an anti-rotation feature can be provided to prevent accidental rotation of needle assembly after pin assembly removal. Such an antirotation feature would prevent the user from accidentally driving the needle further downward and potentially piercing the posterior bone cortex during syringe attachment and removal. Exemplary anti-rotation design configurations include one-way ratcheting of the base to needle assembly after pin removal, and engagement of a spring-loaded brake after pin removal.

While the present embodiments have been described mainly in the context of bone marrow access, it is noted that the presented methods and devices can be used to advance a tissue penetrable needle through a first tissue region having a first tissue region characteristic (such as tissue density, tissue pH, tissue optical properties, etc.) and into a second tissue region having a second tissue region characteristic, wherein the first tissue region characteristic is different than the second tissue region characteristic. As defined in the present application, tissue is broadly defined to include any part of the body, including but not limited to: bones, bone marrow, cartilage, organs, skin and bodily fluids such as blood, air, urine, etc. Further, the second tissue region is defined to also include cavities, such as a bone marrow cavity, a sinus cavity, uterus, stomach or any other cavity within an organ or body part. The various sensor mechanisms described herein can sense a transition of the tissue penetrable needle from the first tissue region into the second tissue region based on the variation in the sensed tissue region characteristic. Thereupon, the sensor mechanisms may automatically stop further advancement of the needle into the second tissue region, or alternatively may alert a user of the device of such transition, whereupon the user may stop further advancement of the needle. Such alerts may include, but are not limited to: an alarm or other auditory signal, a light or other visual signal, a vibration or other tactile signal, etc.

For example, an embodiment using a sensor mechanism comprising a spring-loaded pin can be used when the first tissue density is greater than the second tissue density, and wherein the reduction in tissue density from the first to the second region is sufficient to allow the pin to extend beyond the distal tip of the needle and into the second tissue region. An embodiment using other sensor mechanisms, such as using optical sensors, pH sensors, temperature sensors, electrochemical sensors, etc., can be used when the first and second tissue region characteristics are such that their variation can be sensed by the particular sensor used. As also described above, the sensor mechanisms may be adjusted or tuned in order to configure the device for use at the desired bodily site, based on the tissue region characteristic differential present likely to be encountered at the desired site. Such characteristic differentials may be calculated based on characteristic values of the tissues as known in the art. Thus, the present embodiments allow for automatically stopping advancement further of the needle (or alerting a user) upon detection of any body cavity or more generally any tissue region characteristic change from one tissue region to another, and therefore may be used at other sites such as, but not limited to, the abdomen, lung, skull, spine, kidney, stomach, heart, teeth, bladder, eye, uterus, sinuses, etc.

Furthermore, the present embodiments can be used for industrial applications such as drilling through walls and pipes and drilling for liquid (water, oil, etc.) and air pockets below ground.

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the method and apparatus of the present invention disclosed herein without departing from the spirit and scope of the invention as described here.

1. A tissue access device, comprising:
a catheter assembly with a proximal end, a distal end and an elongated portion therebetween, comprising a tissue penetrable needle at the distal end, wherein the tissue penetrable needle is configured to move through a first tissue region having a first tissue region characteristic and into a second tissue region having a second tissue region characteristic, wherein the first and second tissue region characteristics are different; and
a sensor mechanism at the proximal end, wherein the sensor mechanism is configured to sense transition of the tissue penetrable needle from the first tissue region into the second tissue region.

2. The device of claim 1, wherein the tissue penetrable needle is connected to the proximal end of the catheter by a flexible coil.

3. The device of claim 1, wherein the device is configured to provide an indication of such transition to a user of the device, whereupon the user may stop further advancement of the tissue penetrable needle into the second tissue region.

4. The device of claim 1, wherein the sensor mechanism is a mechanical sensor mechanism.

5. The device of claim 1, wherein the sensor mechanism is configured to stop further advancement of the tissue penetrable needle into the second tissue region upon sensing such transition.

6. The device of claim 5, further comprising:
an actuator configured to engage with the tissue penetrable needle via the sensor mechanism in order to advance the tissue penetrable needle through the first tissue region and into the second tissue region;
wherein the sensor mechanism is configured to stop further advancement of the tissue penetrable needle by disengaging the actuator from the tissue penetrable needle.

7. The device of claim 6, wherein the first tissue region characteristic is a first tissue density and the second tissue region characteristic is a second tissue density.

8. The device of claim 7, wherein the first tissue region is a bone cortex, the second tissue region is a bone marrow cavity, and the tissue penetrable needle is a bone penetrable needle.

9. The device of claim 7, wherein the sensor mechanism is a mechanical sensor mechanism comprising a pin assembly, the pin assembly comprising a spring-loaded pin having a distal tip and extending through the tissue penetrable needle.

10. The device of claim 9, wherein the spring-loaded pin is connected to the pin assembly through a flexible shaft.

11. The device of claim 9, wherein the pin assembly is configured such that advancement of the distal tip of the spring-loaded pin past the distal tip of the needle causes the actuator to disengage from the needle.

12. The device of claim 8, wherein the actuator is rotatable and provides torque for advancing the tissue penetrable needle through the first tissue region and into the second tissue region.

13. The device of claim 7, further comprising a base for placing the device at an access site, wherein the base is configured to receive the tissue penetrable needle and allow the actuator to advance the tissue penetrable needle through tissue and the first tissue region and into the second tissue region.

14. The device of claim 7, wherein the catheter comprises a mechanism near the distal end that limits the force exerted on the tissue by contact of the catheter.

15. The device of claim 14, wherein the force limiting mechanism comprises a spring.

16. The device of claim 7, wherein the catheter comprises a mechanism at the distal end that reduces concentration of force and inhibits inadvertent penetration by the distal tip of the catheter.

17. The device of claim 16, wherein the mechanism comprises a balloon.

18. The device of claim 7, wherein the catheter comprises a vacuum mechanism at the distal end that enables affixing the tip of the catheter to the tissue.

19. The device of claim 7, wherein the catheter comprises a balloon near the distal end that enables anchoring of the tissue access device within a body cavity.

20. The device of claim 7, wherein the device is configured to be insertable through a working channel of a second tissue access device.

21. The device of claim 20, wherein the second tissue access device is a viewing scope or a laparoscopic trocar.

22. A method of accessing a tissue region, comprising:
advancing a tissue penetrable needle of a tissue access device through a first tissue region having a first tissue region characteristic and into a second tissue region having a second tissue region characteristic, wherein the first and second tissue region characteristics are different, and wherein the tissue access device comprises (a) a catheter assembly with a proximal end, a distal end and an elongated portion therebetween; (b) a tissue penetrable needle at the distal end; and (c) a sensor mechanism at the proximal end, wherein the sensor mechanism is configured to sense transition of the tissue penetrable needle from the first tissue region into the second tissue region; and
accessing the second tissue region using the tissue penetrable needle.

23. The method of claim 22, wherein the tissue penetrable needle is connected to the proximal end of the catheter by a flexible coil.

24. The method of claim 22, wherein the device is configured to provide an indication of such transition to a user of the device, whereupon the user may stop further advancement of the tissue penetrable needle into the second tissue region.

25. The method of claim 22, wherein the sensor mechanism is a mechanical sensor mechanism.

26. The method of claim 22, wherein the sensor mechanism is configured to stop further advancement of the tissue penetrable needle into the second tissue region upon sensing such transition.

27. The method of claim 26, wherein the advancing comprises using an actuator, and wherein the sensor mechanism is configured to stop further advancement of the tissue penetrable needle by disengaging the actuator from the tissue penetrable needle.

28. The method of claim 27, wherein the first tissue region characteristic is a first tissue density and the second tissue region characteristic is a second tissue density.

29. The method of claim 28, wherein the first tissue region is a bone cortex, the second tissue region is a bone marrow cavity, and the tissue penetrable needle is a bone penetrable needle.

30. The method of claim 28, wherein the accessing comprises extracting tissue from the second tissue region or injecting a drug into the second tissue region.

31. The method of claim 28, wherein the force exerted on the tissue by the catheter is limited using a force limiting mechanism.

32. The method of claim 31, wherein the force limiting mechanism comprises a spring.

33. The method of claim 28, wherein concentration of force is reduced and inadvertent penetration by the distal tip of the catheter is inhibited using a mechanism.

34. The method of claim 33, wherein the mechanism comprises a balloon.

35. The method of claim 28, wherein a vacuum mechanism at the distal end is used to affix the tip of the catheter to the tissue.

36. The method of claim 28, wherein the device is inserted through the working channel of a viewing scope.
